(19) United States
(12) Patent Application Publication
(10) Pub. No.: US 2015/0119839 A1
(43) Pub. Date: Apr. 30, 2015

(54) QUADRUPED DIAPER WITH EXTENDED REAR LEG COVERS

(71) Applicant: Janet Southward, Buena Park, CA (US)

(72) Inventor: Janet Southward, Buena Park, CA (US)

(21) Appl. No.: 14/064,647

(22) Filed: Oct. 28, 2013

(51) Int. Cl. A61F 13/496

(52) U.S. Cl. CPC A61F 13/496; A61F 2013/15186
USPC 604/385.3

(57) ABSTRACT

A quadruped diaper with extended rear leg covers is disclosed. A diaper of the disclosure includes a textile body band designed to encircle the body of the quadruped, an outer liner extending from the textile body band that is substantially liquid impermeable, wherein the outer liner comprises an extended covering extending from the textile body band and comprising leg portions of sufficient length to cover at least a quarter of a length of each rear leg of the quadruped, an inner liner extending from the textile body band that is substantially liquid permeable, wherein the inner liner comprises an outer perimeter secured to the outer liner to enclose a cavity there between, and an absorbent core retained within the cavity and extending through the leg portions of the diaper.

### TECHNICAL FIELD

The embodiments of the disclosure relate generally to a diaper for canines or other quadrupeds and, more specifically, relate to a quadruped diaper with extended rear leg covers.

### BACKGROUND

Waste discharge by pets in undesirable locations is a problem encountered by pet owners, and in particular, dog (canine) owners. Urination and menstruation with female dogs are particularly messy and difficult to clean. This poses significant problems to pet owners with respect to waste discharge in improper and/or undesirable locations. Current solutions to address waste discharge by pets include variations of diaper-like garments for dogs intended to eliminate one or more of the problems discussed above. These prior solutions are subject to short-comings with respect to effectiveness of fully preventing leaks of the waste discharge from the diaper-like garments.

### BRIEF DESCRIPTION OF THE DRAWINGS

The disclosure will be understood more fully from the detailed description given below and from the accompanying drawings of various embodiments of the disclosure. The drawings, however, should not be taken to limit the disclosure to the specific embodiments, but are for explanation and understanding only.

FIG. 1 is a block diagram illustrating a side view of a quadruped diaper on a canine according to an implementation of the disclosure;

FIG. 2 is a block diagram illustrating another side view of a quadruped diaper on a canine according to an implementation of the disclosure;

FIG. 3 is a block diagram illustrating a front region of a quadruped diaper on a canine according to an implementation of the disclosure; and

FIG. 4 is a block diagram illustrating a back region of a quadruped diaper on a canine according to an implementation of the disclosure.

### DETAILED DESCRIPTION

Embodiments of the disclosure provide for quadruped diaper with extended rear leg covers. While implementations of the disclosure are susceptible of embodiment in many different forms, there is shown in the drawings and will herein be described in detailed specific embodiments with the understanding that the present disclosure is to be considered as an exemplification of the principles of the embodiments and is not intended to limit the implementations to those illustrated.

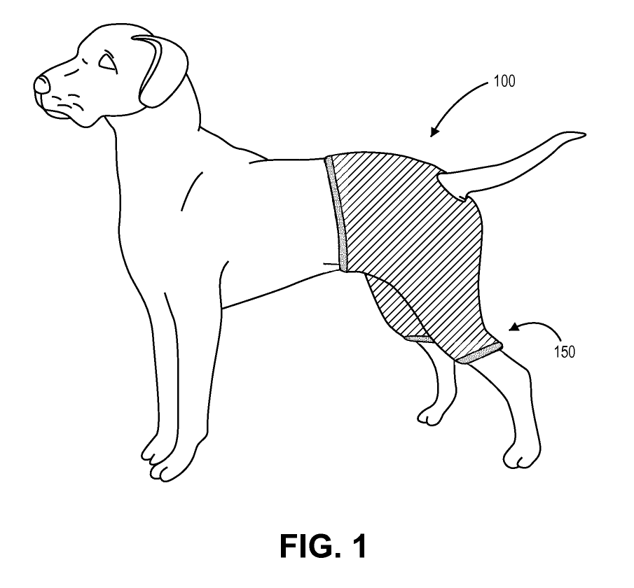

Referring now to FIGS. 1 through 4, views of diaper 100 are shown. Although embodiments refer to a canine diaper, it is envisioning that diaper 100 may also be useful for any type of “quadruped” animal. A quadruped includes an animal that usually moves in a quadrupedal manner (e.g., using four feet).

Diaper 100 generally comprises an outer liner, inner liner, cavity, and an absorbent core. Referring to FIG. 1, in one embodiment, diaper 100 comprises leg portions 150 of sufficient length to cover at least a quarter of a length of each rear leg of a quadruped. Canine diaper 100 may be disposable or reusable. While diaper 100 is shown as having separate liners comprising the same or different materials, which may be fused, fastened, or otherwise attached, it will be understood that a unitary member (liner) is also contemplated. It will be understood that FIGS. 1 through 4 are merely schematic representations of a diaper 100. As such, some of the component may have been distorted from their actual scale for pictorial clarity.

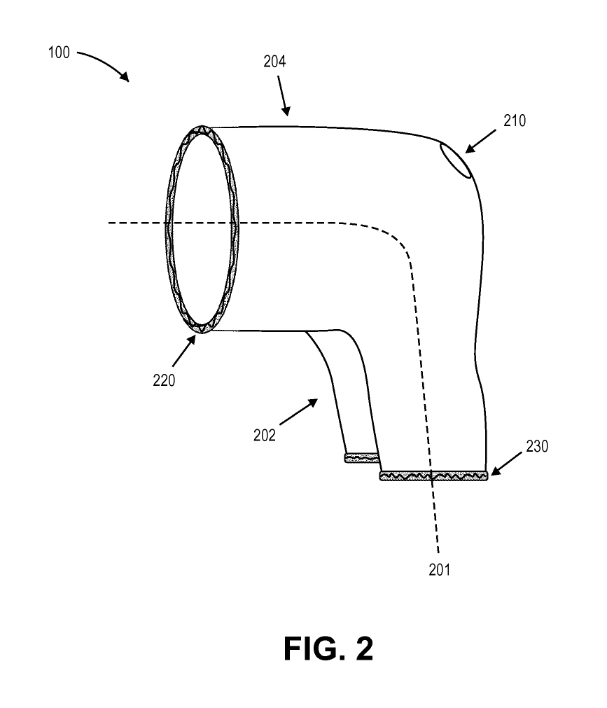

FIG. 2 illustrates diaper 100 as including axis 201, front region 202, back region 204, opening 210, body band 220, and leg ends 230. Axis 201 is shown as extending along a mid-section of the diaper 100 to divide the diaper into the front region 202 and the back region 204. It will be understood that axis 201 is shown for purposes of illustration as referencing the relative position and/or direction of other members of diaper 100.

Front region 202 comprises a region of diaper 100 that is securable about at least a lower or front portion (e.g., stomach) of a torso of the canine. Back region 204 comprises a region of diaper 100 that is securable about at least an upper or rear portion (e.g., back) of a torso of the canine.

Canine diaper 100 may comprise a textile body band 220 designed to encircle the body of the quadruped. The textile body band 220 may form a gathering region around the torso of the canine and be fabricated from, for example, cotton, spandex, elastic, plastic, and/or combinations thereof. However, it will be understood that embodiments are not limited to a particular material of textile body band 220, and that textile body band 220 may be fabricated from any one of a number of natural and/or synthetic materials. In addition, a clip and/or any other fastening component may be utilized in conjunction with textile body band 220 to secure the diaper 100 to the torso of the canine.

Diaper 100 may also include an outer liner extending from the textile body band 220 to provide an extended or elongated covering from the textile body band 220 to leg ends 230 of the diaper 100. The extended covering provided by the outer liner may further comprise leg portions of sufficient length to cover at least a quarter of a length of each rear leg of the canine. In some embodiments, the length may cover more than a quarter of the length of each rear leg. For example, the leg portions may be a length that cover at least a third of each rear leg of the canine.

In one embodiment, the outer liner is substantially liquid impermeable. The outer liner may be made of a decorative textile material, and may be an elastic, flexible material that is flexible and soft for the comfort of the canine. The outer liner may be fabricated from, for example, cotton, spandex, elastic, plastic, and/or combinations thereof. However, it will be understood that embodiments are not limited to a particular material of outer liner, and that outer liner may be fabricated from any one of a number of natural and/or synthetic materials.

Leg ends 230 may form a gathering region around the rear legs of the canine and be fabricated from, for example, cotton, spandex, elastic, plastic, and/or combinations thereof. However, it will be understood that embodiments are not limited to a particular material of leg ends 230, and that leg ends 230 may be fabricated from any one of a number of natural and/or synthetic materials.

Furthermore, diaper 100 may include an inner liner extending from the textile body band 230 that corresponds to the outer liner. For example, the inner liner may comprise an outer perimeter secured (e.g., fused, fastened, or otherwise attached) to the outer liner to enclose a cavity there between. An absorbent core may be retained within the cavity, where the absorbent core extends through the leg portions of the diaper 100 to prevent leakage of waste discharge from the diaper 100.

In one implementation, the inner liner may be substantially liquid permeable. The inner liner may be fabricated from, for example, cotton, spandex, elastic, plastic, and/or combinations thereof. However, it will be understood that embodiments are not limited to a particular material of inner liner, and that inner liner may be fabricated from any one of a number of natural and/or synthetic materials. While diaper 100 is described as having separate liners (inner and outer) comprising the same or different materials, which may be fused, fastened, or otherwise attached, it will be understood that a unitary member (liner) is also contemplated.

In one implementation, diaper 100 includes an opening 210 for a tail of the canine to pass through the diaper 100 for ease of wear of the diaper for the canine. The outer and inner liners, as well as the absorbent core, may include corresponding cutouts for opening 210 of diaper 100.

In some implementations, diaper 100 is fashioned as a seamless diaper into which a canine steps into the diaper from a top opening that comprises the textile body band 210. The diaper 100 may then be then pulled up over the legs and the torso of the canine to securely fasten. In other implementation, the diaper may include a zipper or other fastening component running at least a portion of a length of the back region 204 of diaper 100 to enable ease of putting on and taking off the diaper 100.

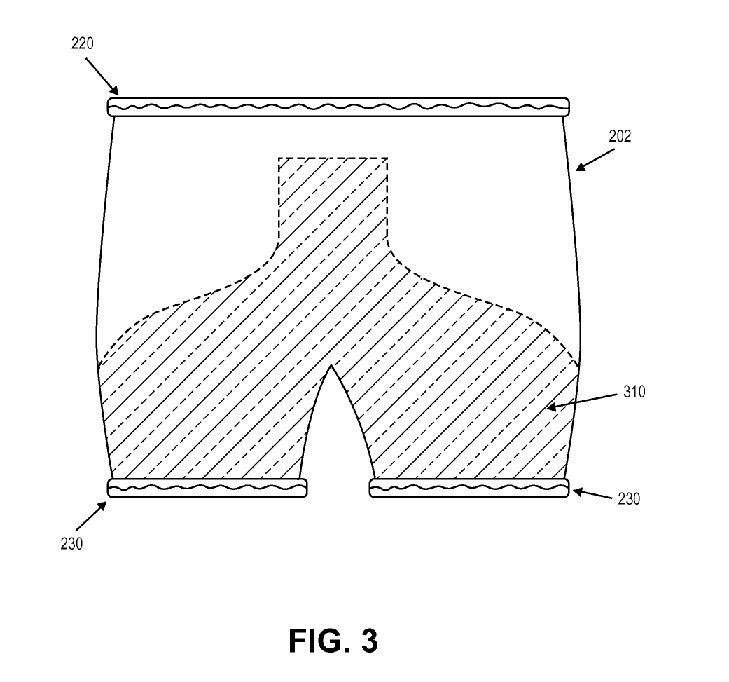

FIG. 3 illustrates a view of front region 202 of diaper 100. Front region 202 includes textile body band 220 and leg ends 230. As shown, front region 202 includes leg portions that extend at least a quarter of a length of each rear leg of a quadruped. Front region 202 also includes absorbent material 310, which also extends into leg portions of diaper 100. This extension of absorbent material 310 into leg portions of diaper 100 aids in preventing leakage of waste discharge from diaper 100.

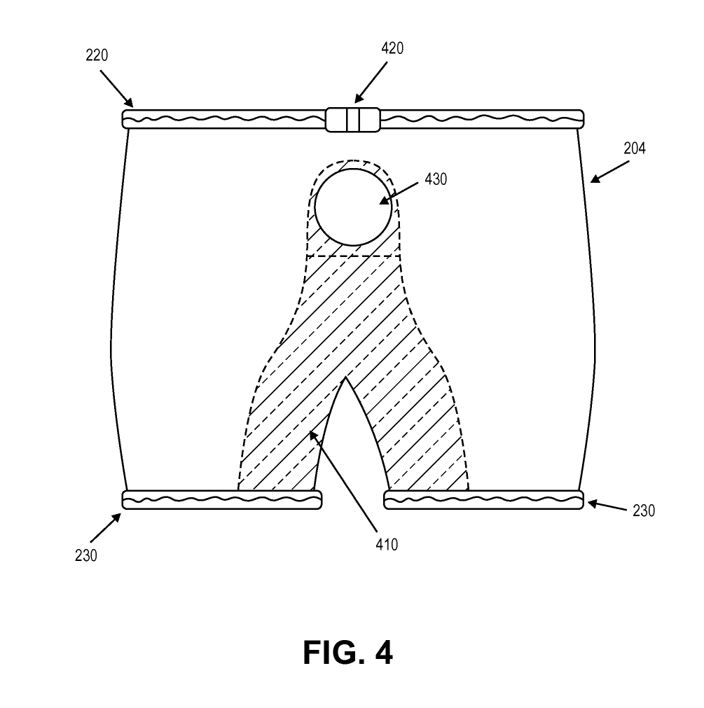

FIG. 4 illustrates a view of back region 204 of diaper 100. Back region 204 includes textile body band 220 and leg ends 230. Textile body band 220 may further comprise a fastening component 420, such as a clip, button, or other securing component to secure the textile body band 220 about the torso of the canine.

As shown, back region 204 includes leg portions that extend at least a quarter of a length of each rear leg of a quadruped. Back region 204 also includes absorbent material 410, which also extends into leg portions of diaper 100 and over, and including, opening 430. In one implementation, opening 430 is the same as opening 210 illustrated with respect to FIG. 2. This extension of absorbent material 310 into leg portions of diaper 100 aids in preventing leakage of waste discharge from diaper 100. It will be understood that diaper 100 and absorbent material 310, 410 may comprise any one of a number of different shapes and sizes of a variety of canines.

In the foregoing description, numerous details are set forth. It will be apparent, however, that the disclosure may be practiced without these specific details. In some instances, well-known structures and devices are shown in block diagram form, rather than in detail, in order to avoid obscuring the disclosure.

Whereas many alterations and modifications of the disclosure will no doubt become apparent to a person of ordinary skill in the art after having read the foregoing description, it is to be understood that any particular embodiment shown and described by way of illustration is in no way intended to be considered limiting. Therefore, references to details of various embodiments are not intended to limit the scope of the claims, which in themselves recite only those features regarded as the disclosure.

What is claimed is:

1. A diaper for a quadruped comprising:
a textile body band designed to encircle the body of the quadruped;
an outer liner extending from the textile body band that is substantially liquid impermeable, wherein the outer liner comprises an extended covering extending from the textile body band and comprising leg portions of sufficient length to cover at least a quarter of a length of each rear leg of the quadruped;
an inner liner extending from the textile body band that is substantially liquid permeable, wherein the inner liner comprises an outer perimeter secured to the outer liner to enclose a cavity there between; and
an absorbent core retained within the cavity and extending through the leg portions of the diaper.

2. The diaper of claim 1, further comprising a fastening device attached to the textile body band to secure the textile body band about the body of the quadruped.

3. The diaper of claim 1, wherein the leg portions comprise an elastic band to secure the leg portions about the rear legs of the quadruped.

4. The diaper of claim 1, further comprising a first opening in the extended section and a second opening in the inner liner that correspond to each other when the inner liner is secured to the outline, the first and second openings for a tail of the quadruped.

5. The diaper of claim 1, wherein the textile body band comprises an elastic band.

6. The diaper of claim 1, wherein the outer liner is constructed of a decorative textile material.

7. The diaper of claim 1, wherein the length of the leg portions cover at least a third of a length of each rear leg of the quadruped.

8. The diaper of claim 1, wherein the quadruped is a canine.
